(19) United States
(12) Patent Application Publication
(10) Pub. No.: US 2011/0171324 A1
(43) Pub. Date: Jul. 14, 2011

(54) METHOD OF TREATING OR PREVENTING DISEASE IN BEES

(76) Inventor: Richard William Clemente, Westford, MA (US)

(21) Appl. No.: 12/687,462

(22) Filed: Jan. 14, 2010

(51) Int. Cl. A61K 33/14; A61P 43/00

(52) U.S. Cl. 424/680

(57) ABSTRACT

The invention is directed to a method of a method of treating or preventing disease in honeybees, a method of promoting the health of honey bees and to a method of increasing the production of honeybees in a hive or colony.

### BACKGROUND OF THE INVENTION

Species of bees used for honey production throughout the world include Apis mellifera, Apis cerana and Apis florae. Apis mellifera is the species of honeybee present in North America. In addition to providing honey, honeybees are a major source of pollination for numerous commercial crops in the United States. Numerous diseases and infestations threaten beekeeping, cause decreased honey production and decreased crop pollination. These diseases include, for example colony collapse disorder and Varroasis. Varroasis is a parasitic disease caused by the Varroa mite (Varroa mite species include Varroa destructor and Varroa jacobsoni mites). The Varroa mite infests bees at the larval stage and can pass between honeybees with social contact. Varroa mite infestation in bee hives decreases honey production and decreases winter survival. A colony infected with Varroa mites can be killed off within 1 to 2 years. Varroa mite infestation is believed to be a contributing factor in colony collapse disorder (van Engelsdorp D et al., (2009) Colony Collapse Disorder: A Descriptive Study. PLoS ONE 4(8): e6481. doi:10.1371/journal.pone.0006481).

Various pesticides including, for example, the pyrethroid (APISAN®) and coumaphos, (CHECK-MITE®) can be used to reduce Varroa mite infestation. However, currently available pesticides are not entirely effective at eradicating mite infestation and are also associated with the risks of toxicity to the honeybees themselves as well as the risk contaminating the produced honey. It would therefore be advantageous to devise additional methods of treating or preventing diseases associated with Varroa mite infestation in honeybees.

### SUMMARY OF THE INVENTION

The present invention is based on the surprising discovery that administering to bees a solution comprising sodium chloride and other minerals results in a dramatic decrease in Varroa mite infestation and increases winter survival. For example, Example 1 shows that it was observed that winter survival of bees increased and Varroa mite infestation was not detected in bee hives that were provided with an aqueous solution of sodium chloride and other specific minerals.

In one aspect, the invention is directed to a method of treating or preventing disease in honeybees comprising administering to said honeybees an effective amount of sodium chloride. In another aspect of the invention, the bees are administered an effective amount of sodium chloride and an effective amount of at least one mineral selected from the group consisting of iodine, copper, magnesium, manganese, iron, sulfur, cobalt, and zinc. In certain aspects, the disease is associated with an acarine, fungal or bacterial infestation of a bee hive or colony. In additional embodiments, the disease is associated with an acarine infestation. In a further embodiment, the disease is associated with Varroa mite infestation. In a further embodiment, the disease is colony collapse disorder.

In certain aspects of the invention, the sodium chloride and at least one mineral selected from the group consisting of iodine, copper, magnesium, manganese, iron, sulfur, cobalt, and zinc are administered in aqueous solution. In some aspects, the sodium chloride and at least one mineral are administered together in an aqueous solution. In one embodiment, the aqueous solution comprises an amount of sodium chloride from about 0.5 to about 5 g per gallon of water.

In another embodiment, the invention is a method of promoting the health of honeybees comprising administering to said honeybees an effective amount of sodium chloride and at least one mineral selected from the group consisting of iodine, copper, magnesium, manganese, iron, sulfur, cobalt, and zinc. In some aspects, the sodium chloride and at least one mineral are administered in an aqueous solution.

In yet another embodiment, the invention is a method of increasing winter survival of honey bees comprising administering to said honeybees an effective amount of sodium chloride and at least one mineral selected from the group consisting of iodine, copper, magnesium, manganese, iron, sulfur, cobalt, and zinc. In some aspects, the sodium chloride and at least one mineral are administered in an aqueous solution.

In a further embodiment, the invention is a method of increasing the production of honey of a colony or a hive and/or increasing the amount of honey obtained from a colony or hive comprising administering to said honeybees an effective amount of sodium chloride and at least one mineral selected from the group consisting of iodine, copper, magnesium, manganese, iron, sulfur, cobalt, and zinc. In some aspects, the sodium chloride and at least one mineral are administered in an aqueous solution.

In yet another embodiment, the invention is an acaracidic composition comprising from about 0.4 to about 0.8% (w/v) sodium chloride in an aqueous solution, wherein the solution further comprises a mineral selected from the group consisting iodine, copper, magnesium, manganese, iron, sulfur, cobalt, and zinc.

### DETAILED DESCRIPTION OF THE INVENTION

A description of preferred embodiments of the invention follows.

The words “a” or “an” are meant to encompass one or more, unless otherwise specified.

“Treating” or “treatment” includes the administration of the compositions or agents described herein to prevent or delay the onset of the symptoms, or biochemical indicia of a disease, alleviating or ameliorating the symptoms or arresting or inhibiting further development of the disease, condition, or disorder. The terms “treating” or “treatment” encompass preventing or prevention of disease.

An “effective amount” is an amount which, alone or in combination with one or more other agents, can control, decrease, inhibit, ameliorate, prevent or otherwise affect one or more symptoms of a disease or condition to be treated. For example, an effective amount sodium chloride and an additional mineral encompasses an amount of sodium chloride and an amount of other mineral wherein the combined amounts are sufficient to affect one or more symptoms of a disease or condition to be treated.

The term “administering” as used herein encompasses feeding and/or causing honeybees to ingest or consume.

The term “honeybee” includes, for example, the species Apis mellifera and Apis cerana.

A bee hive is a cavity occupied by a honeybee colony. A colony is a community of bees including a queen and thousands of worker bees.

Diseases that can be treated or prevented using the methods described herein include those caused or associated with acarine infestation, bacterial infestation and fungal infestation. Acarine infestation includes infestation with Varroa mites, Acarapis woodii and Tropilaelaps clareae. The term “Varroa mites” encompass Varraoa destructor and Varroa jacobsoni species. Exemplary bacterial infestations are those caused or associated with Bacillus larvae and/or Melissococcus pluton. Foulbrood disease is a disease caused or associated with Bacillus larvae and/or Melissococcus pluton. Exemplary fungal infestations are those caused or associated with Ascophaera apis infestation. An exemplary disease caused or associated with fungal infestation is Chalkbrood disease. An additional disease that can be treated or prevented using the methods described herein includes colony collapse disorder.

The phrase “promoting the health” refers to reducing the number of deaths in a colony or hive, reducing symptoms of disease and/or increasing honey production. The phrase “increasing winter survival” refers to decreasing the number of bee deaths per hive or colony during a winter season.

The methods described herein comprise administration of an effective amount of sodium chloride, optionally in an aqueous solution. In some aspects, the methods comprise administration of an effective amount of sodium chloride and at least one mineral selected from the group consisting of iodine, copper, magnesium, manganese, iron, sulfur, cobalt, and zinc. In another embodiment, the method comprises administration of an effective amount of sodium chloride and at least two minerals selected from the group consisting of iodine, copper, magnesium, manganese, iron, sulfur, cobalt, and zinc. In yet another embodiment, the method comprises administration of an effective amount of sodium chloride and at least three minerals selected from the group consisting of iodine, copper, magnesium, manganese, iron, sulfur, cobalt, and zinc. In a further embodiment, the method comprises administration of an effective of amount of sodium chloride and at least four minerals selected from the group consisting of iodine, copper, magnesium, manganese, iron, sulfur, cobalt, and zinc. In an additional embodiment, the method comprises administering an effective of amount of a composition comprising sodium chloride, iodine, copper, magnesium, manganese, iron, sulfur, cobalt, and zinc.

In a further aspect of the invention, the methods comprise administration of an effective amount of sodium chloride and at least one mineral selected from the group consisting of manganous oxide, ferrous carbonate, magnesium oxide, calcium sulfate, copper oxide, cobalt carbonate and zinc oxide. In another embodiment, the method comprises administration of an effective amount of sodium chloride and at least two minerals selected from the group consisting of manganous oxide, ferrous carbonate, magnesium oxide, calcium sulfate, copper oxide, cobalt carbonate and zinc oxide. In yet another embodiment, the method comprises administration of an effective amount of sodium chloride and at least three minerals selected from the group consisting of manganous oxide, ferrous carbonate, magnesium oxide, calcium sulfate, copper oxide, cobalt carbonate and zinc oxide. In a further embodiment, the method comprises administration of an effective of amount of sodium chloride and at least four minerals selected from the group consisting of manganous oxide, ferrous carbonate, magnesium oxide, calcium sulfate, copper oxide, cobalt carbonate and zinc oxide. In an additional embodiment, the method comprises administering an effective of amount of sodium chloride, manganous oxide, ferrous carbonate, magnesium oxide, calcium sulfate, copper oxide, cobalt carbonate and zinc oxide.

In some aspects, the method comprises administration of sodium chloride in an aqueous solution wherein the sodium chloride is present in an amount from about 0.5 to about 5 g sodium chloride per gallon of water. In another embodiment, the sodium chloride is present in an amount from about 1 to about 3 g sodium chloride per gallon of water. In a further aspect, the sodium chloride is present in an amount from about 2 to about 3 g sodium chloride per gallon of water.

The method also comprises administration of sodium chloride in an aqueous solution, wherein the sodium chloride is present in an amount from about 0.5 to about 5 g, or from about 1 to about 3 g, or from about 2 to about 3 g sodium chloride per gallon of water and one or more minerals listed in Table A below in the following amounts (milligram mineral per gallon of water).

TABLE A

| Mineral | Weight (mg) per gallon |
|---|---|
| Manganese | About 2 to about 7 |
| Iron | About 0.5 to about 4.5 |
| Magnesium | About 0.5 to about 4.5 |
| Sulfur | About 0.05 to about 3 |
| Copper | About 0.1 to about 0.7 |
| Cobalt | About 0.05 to about 0.7 |
| Zinc | About 0.02 to about 0.3 |
| Iodine | About 0.01 to about 0.3 |

In another aspect, the method comprises administration of sodium chloride in an aqueous solution, wherein the sodium chloride is present in an amount from about 0.5 to about 5 g, or from about 1 to about 3 g, or from about 2 to about 3 g sodium chloride per gallon of water and one or more minerals listed in Table B below in the following amounts.

TABLE B

| Mineral | Weight (mg) per gallon |
|---|---|
| Manganese | About 4 to about 5 |
| Iron | About 1.8 to about 2.8 |
| Magnesium | About 1.8 to about 2.8 |
| Sulfur | About 0.7 to about 1.7 |
| Copper | About 0.1 to about 0.7 |
| Cobalt | About 0.4 to about 0.5 |
| Zinc | About 0.17 to about 0.22 |
| Iodine | About 0.11 to about 0.21 |

In another aspect, the method comprises administration of sodium chloride in an aqueous solution, wherein the sodium chloride is present in an amount from about 0.5 to about 5 g, or from about 1 to about 3 g, or from about 2 to about 3 g sodium chloride per gallon of water and one or more minerals listed in Table C below in the following amounts.

TABLE C

| Mineral | Weight (mg) per gallon |
|---|---|
| Manganese | About 4.6 |
| Iron | About 2.3 |
| Magnesium | About 2.3 |
| Sulfur | About 1.2 |
| Copper | About 0.47 |
| Cobalt | About 0.23 |
| Zinc | About 0.19 |
| Iodine | About 0.16 |

It is to be understood that the methods described herein can comprise administration of sodium chloride and one, two, three, four or more of the minerals described above in Tables A, B or C.

As will be understood by one of skill in the art, the amount of inventive aqueous mineral solution provided to a hive or colony for a given period of time (such as a week, two weeks or a month) varies depending on a number of factors including, for example, the number of hives, and water evaporation. In one example, a gallon of water comprising an effective amount of sodium chloride and an optional additional mineral described herein would be sufficient for one hive for one week. In another example, a gallon of water comprising an effective amount of sodium chloride and an optional additional mineral described herein would be sufficient for one hive for one month. In yet another example, 2 gallons of water comprising an effective amount of sodium chloride and an optional additional mineral described herein would be sufficient for three hives for one week. The aqueous mineral solution can be provided in any appropriate container and can be placed in the vicinity of a bee hive or bee colony.

The invention also encompasses an acaracidic composition comprising from about 0.4 to about 0.8 grams (g)/liter (1) sodium chloride in an aqueous solution, wherein the solution further comprises at least one mineral selected from the group consisting iodine, copper, magnesium, manganese, iron, sulfur, cobalt, and zinc. In some aspects, the composition comprises 0.4 to 0.8 g/l sodium chloride in an aqueous solution, wherein the solution comprises at least two minerals selected from the group consisting of iodine, copper, magnesium, manganese, iron, sulfur, cobalt, and zinc. In other aspects, the composition comprises 0.4 to 0.8 g/l sodium chloride in an aqueous solution, wherein the solution comprises at least three minerals selected from the group consisting of iodine, copper, magnesium, manganese, iron, sulfur, cobalt, and zinc. In yet other aspects, the composition comprises 0.4 to 0.8 g/l sodium chloride in an aqueous solution, wherein the solution comprises at least four minerals selected from the group consisting of iodine, copper, magnesium, manganese, iron, sulfur, cobalt, and zinc.

In another embodiment, the composition comprises from about 0.54 to about 0.64 g/l sodium chloride in an aqueous solution, wherein the solution further comprises at least one mineral selected from the group consisting iodine, copper, magnesium, manganese, iron, sulfur, cobalt, and zinc. In some aspects, the composition comprises 0.54 to about 0.64 g/l sodium chloride in an aqueous solution, wherein the solution comprises at least two minerals selected from the group consisting of iodine, copper, magnesium, manganese, iron, sulfur, cobalt, and zinc. In other aspects, the composition comprises 0.54 to about 0.64 g/l sodium chloride in an aqueous solution, wherein the solution comprises at least three minerals selected from the group consisting of iodine, copper, magnesium, manganese, iron, sulfur, cobalt, and zinc. In yet another aspects, the composition comprises 0.54 to about 0.64 g/l sodium chloride in an aqueous solution, wherein the solution comprises at least four minerals selected from the group consisting of iodine, copper, magnesium, manganese, iron, sulfur, cobalt, and zinc.

In a further aspect, the composition comprises 0.4 to 0.8 g/l sodium chloride, or from about 0.54 to about 0.64 g/l sodium chloride one or more minerals listed in Table D below in the following amounts (milligram mineral per liter of water).

TABLE D

| Mineral | Weight (mg) per liter |
|---|---|
| Manganese | About 0.74 to about 1.74 |
| Iron | About 0.3 to about 0.9 |
| Magnesium | About 0.3 to about 0.9 |
| Sulfur | About 0.1 to about 0.5 |
| Copper | About 0.08 to about 0.32 |
| Cobalt | About 0.05 to about 0.08 |
| Zinc | About 0.03 to about 0.07 |
| Iodine | About 0.03 to about 0.07 |

It is to be understood that the method described herein can comprise administration of sodium chloride and one, two, three, four or more of the minerals described above in Table D.

The following Example further illustrates the present invention but should not be construed as in any way limiting its scope.

### Example 1

### Providing Bee Hives with a Mineral Solution Decreased Varroa Mite Infestation and Increases Winter Survival

A 4 lb (1.8 kg) Red Trace Mineral Salt Brick (Roto Salt Company, NY) was pulverized into a powder. ½ teaspoons of powder was added to 1 gallon of water and the 1 gallon solution was provided in a container outside the beehive in which no pesticides (for example, no Apistan or coumaphos) were used. The solution was replenished as needed (as it was consumed or as it evaporated) from early Spring to late Fall.

Varroa mite infestation was monitored for three consecutive months (July to September) by taking 100 adult bees and placing them in a Mason jar followed by the addition of powdered sugar. Powdered sugar was added in an amount that filled the jar about 1 inch from the bottom of the jar. Bee samples were taken from three bee hives. A screen mesh was placed over the opening of each jar and the jar was shaken vigorously for about 45 seconds to distribute the sugar over the bees. Each jar was then inverted onto a surface until all it appeared that all of the sugar had been removed from the jar. The bees were removed from the jar. The number of mites in the powdered sugar on the surface was then counted. No mites were observed in any of the three hives for which the mineral solution was provided. This experiment was repeated three times in each of the three months during which mite infestation was monitored and at each time, no mites were observed. That no mites were observed in the powdered sugar test was surprising since Applicant has generally observed in previous seasons (during which the mineral solution was not provided), about 1-2 mites in bee samples of 100 bees. Since there are about 60,000 to about 80,000 bees per hive, 1-2 mites per 100 bee samples translates to about 600 to 1600 mites per hive.

Bee death was also monitored during the winter season (about November to March) for the hives that had been provided mineral solution during the Spring to Fall seasons. For two successive winters, death was monitored by counting the number of dead bees on the bottom board of the hive. Less than four dead bees were observed during the two winter seasons for which bee death was monitored. This result was surprising since Applicant has observed in previous winter seasons (where bee hives were not provided with a mineral solution during early Spring to late Fall), that at least 50 to 300 bees per hive died during the winter season.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating or preventing disease in honeybees comprising administering to said honeybees an effective amount of sodium chloride and at least one mineral selected from the group consisting of iodine, copper, magnesium, manganese, iron, sulfur, cobalt, and zinc.

2. The method of claim 1, wherein the disease is a parasitic disease.

3. The method of claim 1, wherein the disease is colony collapse disorder.

4. The method of claim 1, wherein said minerals are administered in an aqueous solution.

5. The method of claim 1, wherein the parasitic disease is caused by or associated with Varroa mite infestation.

6. The method of claim 1, wherein the solution comprises sodium chloride and at least two minerals are selected from the group consisting of iodine, copper, magnesium, manganese, iron, sulfur, cobalt, and zinc.

7. The method of claim 1, wherein the solution comprises sodium chloride and at least three minerals selected from the group consisting of iodine, copper, magnesium, manganese, iron, sulfur, cobalt, and zinc.

8. The method of claim 1, wherein the solution comprises sodium chloride, and a mineral selected from the group consisting of manganous oxide, ferrous carbonate, magnesium oxide, calcium sulfate, copper oxide, cobalt carbonate and zinc oxide.

9. The method of claim 2, wherein the solution comprises from about 0.5 to about 5 g sodium chloride per gallon of water.

10. The method of claim 9, wherein the solution comprises from about 1 to about 3 g sodium chloride per gallon of water.

11. The method of claim 10, wherein the solution comprises from about 2 to about 3 g sodium chloride per gallon of water.

12. The method of claim 1, wherein the solution comprises sodium chloride, iodine, copper, magnesium, manganese, iron, sulfur, cobalt, and zinc.

13. The method of claim 4, wherein the solution comprises sodium chloride, iodine, copper, magnesium, manganese, iron, sulfur, cobalt, and zinc.

14. A method of increasing the winter survival of honeybees comprising administering to said honeybees an effective amount of sodium chloride and at least one mineral selected from the group consisting of iodine, copper, magnesium, manganese, iron, sulfur, cobalt, and zinc.

15. The method of claim 14, wherein said minerals are administered in an aqueous solution.

16. The method of claim 15, wherein the solution comprises sodium chloride and at least two minerals are selected from the group consisting of iodine, copper, magnesium, manganese, iron, sulfur, cobalt, and zinc.

17. The method of claim 16, wherein the solution comprises from about 0.5 to about 5 g sodium chloride per gallon of water.

18. A method of increasing the production of honey comprising administering honeybees an effective amount of sodium chloride and at least one mineral selected from the group consisting of iodine, copper, magnesium, manganese, iron, sulfur, cobalt, and zinc.

19. The method of claim 17, wherein said minerals are administered in an aqueous solution.

20. The method of claim 18, wherein the solution comprises from about 0.5 to about 5 g sodium chloride per gallon of water.
